(19) United States
(12) Patent Application Publication
(10) Pub. No.: US 2016/0175222 A1
(43) Pub. Date: Jun. 23, 2016

(54) WATERPROOF MASCARA COMPOSITION

(71) Applicant: L'OREAL, Paris (FR)

(72) Inventors: Florentina Mihaela PAVEL, Hillsborough, NJ (US); Angeles FONOLLA-MORENO, Scotch Plains, NJ (US)

(73) Assignee: L'OREAL, Paris (FR)

(21) Appl. No.: 14/573,865

(22) Filed: Dec. 17, 2014

(51) Int. Cl. A61K 8/60; A61Q 1/10

(52) U.S. Cl. CPC A61K 8/604; A61Q 1/10; A61K 2800/40

(57) ABSTRACT

A mascara composition including an oxyalkylenated ether of a sugar fatty acid ester.

### TECHNICAL FIELD

The present invention generally relates to a cosmetic composition for keratinous materials such as keratin fibers, in particular, eyelashes and eyebrows, more particularly eyelashes. The present invention also generally relates to a method of making up eyelashes.

### BACKGROUND

Waterproof mascara compositions may attract consumers because of their long-lasting, non-transfer properties. However, conventional waterproof cosmetic compositions are generally difficult to remove, and require special cleansing products such as makeup removers to completely remove from the skin or keratin fibers. Such makeup removers may make the consumers feel uncomfortable upon application onto the skin or keratin fibers because of their smell, feel, and texture. Also, use of makeup removers can be damaging to eyelashes, causing eyelash loss during removal of mascara composition from the eyelashes. In addition, the consumers who do not have the makeup removers may be required to purchase an additional product just to remove waterproof mascara compositions.

Thus, there is a need for a waterproof mascara composition which does not have the above-described drawbacks of the conventional waterproof mascara compositions. In particular, there is a strong need for a waterproof mascara composition which can be removed easily from keratinous materials, preferably without the need of special cleansing products.

Accordingly, one aspect of the present invention is a mascara composition which is able to address or overcome at least one of the aforementioned problems with the prior art products. In particular, one aspect of the present invention is a mascara composition which is able to be removed with water with mild rubbing.

### SUMMARY

One aspect of the present invention relates to a mascara composition including at least one oxyalkylenated ether of a sugar fatty acid ester. Preferably, the mascara composition is not in the form of an emulsion. Preferably, the amount of the oxyalkylenated ether of a sugar fatty acid ester is at least about 3% by weight, preferably at least about 5% by weight, relative to the total weight of the mascara composition.

Another aspect of the present invention relates to a method of making up keratinous materials including applying a mascara composition including at least one oxyalkylenated ether of a sugar fatty acid ester onto keratinous materials in an amount sufficient to make up the keratinous materials. Preferably, the mascara composition is not in the form of an emulsion.

Another aspect of the present invention relates to a method of enhancing an appearance of eyelashes including applying a mascara composition including at least one oxyalkylenated ether of a sugar fatty acid ester onto the eyelashes in an amount sufficient to enhance the appearance of the eyelashes. Preferably, the mascara composition is not in the form of an emulsion.

Another aspect of the present invention relates to a method of improving removal properties of a waterproof cosmetic composition, including mixing at least one oxyalkylenated ether of a sugar fatty acid ester with the other ingredients of a waterproof cosmetic composition to form a final mascara composition. Preferably, the mixture composition is not in the form of an emulsion.

Another aspect of the present invention relates to a process of making a mascara composition including mixing at least one oxyalkylenated ether of a sugar fatty acid ester and a solvent to form a mascara composition. Preferably, the mascara composition is not in the form of an emulsion.

It is to be understood that both the foregoing general description and the following detailed description are exemplary and explanatory only, and are not restrictive of the invention.

### DETAILED DESCRIPTION

Unless otherwise indicated, all numbers expressing quantities of ingredients and/or reaction conditions are to be understood as being modified in all instances by the term “about.” Accordingly, unless indicated to the contrary, the numerical parameters set forth in the following specification and attached claims are approximations that may vary depending upon the desired properties sought to be obtained by the present invention.

As used herein, the expression “at least one” means one or more and thus includes individual components as well as mixtures/combinations.

As used herein, the singular forms “a,” “an,” and “the” are intended to include the plural forms as well, unless the context clearly indicates otherwise. It will further understood that the terms “comprising,” “including,” and variants thereof, when used in this specification, specify the presence of stated features, elements, and/or components, but do not preclude the presence or addition of one or more other features, elements, components, and/or groups thereof

As used herein, “mascara” and “mascara composition” mean a composition that is intended to be applied to keratinous materials, preferably keratin fibers, in particular eyelashes and/or eyebrows.

As used herein, “keratinous materials” include, but are not limited to, skin, nail, living keratin fibers such as head hair, eyelashes, and eyebrows, and non-living keratin fibers such as swatches, extensions, and false eyelashes. The living and non-living keratin fibers include any mammalian hair, including human hair.

As used herein, “waterproof” refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc. In a preferred embodiment of the present invention, little or no composition is transferred from the wearer.

As used herein, “water-removable” or “removable with water” mean that a majority of a composition can be removed from the surface on which the composition is applied with water and soap, preferably with water alone, with mild rubbing. A water-removable composition preferably does not require use of oils for removal.

As used herein, a “long-lasting” composition refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

The mascara composition and methods of the present invention can comprise, consist of, or consist essentially of the essential elements and limitations of the invention described herein, as well as any additional or optional ingredients, components, or limitations described herein or otherwise useful in personal care compositions intended for application to keratinous materials. For purposes of the mascara composition of the present invention, the basic and novel property of a mascara composition “consisting essentially of” at least one oxyalkylenated ether of a sugar fatty acid ester is to improve the water-removable properties of the mascara composition.

(Oxyalkylenated ether of a sugar fatty acid ester)

According to one aspect of the present invention, the mascara composition includes at least one oxyalkylenated ether of a sugar fatty acid ester. The oxyalkylenated ether of a sugar fatty acid ester may include from about 1 to about 200, preferably from about 10 to about 100, more preferably from about 15 to about 50, moles of an oxyalkylenated group, including all ranges and subranges therebetween. In a preferred embodiment of the present invention, the oxyalkylenated ether of a sugar fatty acid ester has about 1 to about 200, preferably from about 10 to about 100, more preferably from about 15 to about 50 moles of ethylene oxide, propylene oxide, or mixtures thereof, including all ranges and subranges therebetween. Preferably, the oxyalkylenated ether of a sugar fatty acid ester has about 1 to about 200, preferably from about 10 to about 100, more preferably from about 15 to about 50 moles of ethylene oxide, including all ranges and subranges therebetween.

The sugar fatty acid ester may be an ester of various types of fatty acids and of sugars, as far as the ester is cosmetically or dermatologically acceptable. As used herein, “cosmetically acceptable” or “dermatologically acceptable” is intended to mean that a composition is suitable for use in contact with human tissues such as keratinous materials and mucous membranes without undue toxicity, incompatibility, instability, and/or allergic response.

The sugar fatty ester is preferably a mono- or di-ester of a sugar. “Sugar,” as used herein, is intended to include oxygen-containing hydrocarbon compounds that possess several alcohol functions, with or without aldehyde or ketone functions, and having at least 4 carbon atoms. These sugars can be monosaccharides, oligosaccharides or polysaccharides.

As suitable sugars, non-limiting examples include sucrose, glucose, galactose, ribose, fucose, maltose, fructose, mannose, arabinose, xylose, lactose, and their derivatives, for example alkylated, such as methylated derivatives such as methylglucose. Preferably, the sugar is glucose or its alkylated derivative such as an alkylglucose. As used herein, (alkyl)glucose is intended to include glucose, an alkylglucose, and a mixture of glucose and an alkylglucose.

The esters of sugars and of fatty acids are preferably chosen from the esters or mixtures of esters of sugars described above and of linear or branched, saturated or unsaturated C₆-C₃₀, such as C₁₂-C₂₂, fatty acids. If they are unsaturated, these compounds can have one to three, conjugated or unconjugated, carbon-carbon double bonds.

The esters may also be chosen from mono-, di-, tri- and tetra-esters, polyesters and mixtures thereof.

These esters can be, for example, oleate, laurate, palmitate, myristate, behenate, cocoate, stearate, linoleate, linolenate, caprate, arachidonates, or mixtures thereof, such as the oleo-palmitate, oleo-stearate, palmito-stearate mixed esters.

For example, in the present invention, the mono- and di-esters may be used. Preferably sugar fatty acid esters may include the mono- or di-oleate, stearate, behenate, oleopalmitate, linoleate, linolenate, oleostearate, of sucrose, of glucose, or of methylglucose.

The oxyalkylenated ether of a sugar fatty acid esters that may preferably be used in the present invention include, for example, oxyethylenated ethers of a fatty acid and methylglucose, such as a polyethylene glycol ether of a diester of methylglucose and stearic acid containing about 20 moles of ethylene oxide (INCI name: PEG-20 methyl glucose distearate), a polyethylene glycol ether of a mixture of the monoester and diester of methyl glucose and stearic acid containing about 20 moles of ethylene oxide (1NCI name: PEG-20 methyl glucose sesquistearate, such as Glucamate® SSE-20 sold by Lubrizol). These oxyalkylenated ethers may be used alone or in combination.

According to one preferable aspect of the present invention, the oxyalkylenated ether of a sugar fatty acid ester is an ethoxylated (alkyl)glucose fatty acid ester. Among ethoxylated (alkyl)glucose fatty acid esters, an ethoxylated (alkyl)glucose stearate, in particular, PEG-20 methyl glucose sesquistearate is preferred.

The amount of the oxyalkylenated ether of a sugar fatty acid ester is preferably at least about 3% by weight, more preferably at least about 5% by weight, relative to the total weight of the mascara composition. Preferably, the mascara composition includes the oxyalkylenated ether of a sugar fatty acid ester(s) in an amount from about 3% to about 15% by weight, more preferably from about 5% to about 10% by weight, relative to the total weight of the mascara composition, including all ranges and subranges therebetween.

### (Solvents)

The mascara composition may optionally include at least one volatile solvent, and may further optionally include at least one non-volatile solvent. Suitable non-volatile or volatile solvents can be selected based on the intended use. As used herein, the term “non-volatile” means having a boiling point of greater than about 100° C.

The non-volatile solvent typically includes at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol® 810, 812 and 818 by the company Dynamit Nobel;
synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, with R₆+R₇≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol; and
mixtures thereof.

The amount of the non-volatile solvent, if present, is preferably less than about 1% by weight, such as from about 0.01% to about 0.5% by weight, including all ranges and subranges therebetween, relative to the total weight of the mascara composition.

The volatile solvent typically includes at least one volatile oil. The volatile oil may be a volatile silicone oil or a volatile non-silicone oil.

Suitable volatile silicone oils include, but are not limited to, linear or cyclic silicone oils having a viscosity at room temperature less than or equal to 6cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

Suitable volatile non-silicone oils may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

Non-limiting examples of volatile non-silicone oils are listed in Table 2 below.

TABLE 2

| Compound | Flash Point (° C.) |
|---|---|
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

In general, the at least one volatile solvent, if present, is present in the composition in an amount of from about 5% to about 80% by weight, such as from about 10% to about 60% by weight, and from about 20% to about 40% by weight, relative to the total weight of the mascara composition, including all ranges and subranges therebetween.

### (Colorants)

According to the present invention, the mascara compositions may optionally include at least one colorant. Suitable colorants include, but are not limited to, pigments, dyes such as liposoluble dyes, nacreous pigments, and pearling agents. Typically, when the composition contains colorants, the composition may be used as a mascara composition. Alternatively, when the composition does not contain colorants, it is a clear or transparent composition which can be used as a basecoat (or topcoat) prior to (or after) application of a mascara composition to keratinous materials. However, it is possible that topcoats or basecoats could contain colorants, and/or that a mascara composition could contain little or no colorant.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration of up to about 20% by weight of the total weight of the mascara composition, such as from about 0.0001% to about 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, is in the composition in a concentration of up to about 50% by weight relative to the total weight of the mascara composition, such as from about 0.1% to about 20% by weight, preferably from about 0.1% to about 15% by weight, including all ranges and subranges therebetween.

Representative pigments which may be used according to the present invention include white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum. If present, the pigments may be in the mascara composition in a concentration of up to about 50% by weight of the total weight of the mascara composition, such as from about 0.5% to about 40% by weight, and further such as from about 2% to about 30% by weight, including all ranges and subranges therebetween. In the case of certain products, the pigments, including nacreous pigments, may, for example, represent up to about 50% by weight of the mascara composition.

(Additional ingredients)

The mascara composition of the present invention may further include various additives desirably used in cosmetic or dermatological compositions. For example, dispersants, anti-oxidants, pH adjusters, preservatives, neutralizing agents, fragrances, fillers, film formers, co-solvents, waxes, plasticizers, cosmetic and dermatological active agents such as emollients, moisturizers, vitamins, UV filters, and sunscreens, and mixtures thereof can be added. A non-exhaustive listing of such ingredients can be found in the CTFA International Cosmetic Ingredient Dictionary and Handbook, Fourteenth Edition (2012), contents of which are incorporated herein by reference in its entirety.

One skilled in the art will take care to select the optional additional additives and/or the amount thereof such that the advantageous properties of the mascara compositions according to the present invention are not, or are not substantially, adversely affected by the envisaged addition.

These substances may be selected variously by one skilled in the art to prepare a composition which has the desired properties, for example, consistency or texture.

The mascara composition of the present invention may include water. The amount of water is preferably less than 5% by weight relative to the total weight of the mascara composition. More preferably, the mascara composition is substantially free of water, further more preferably completely free of water. As used herein, “substantially free of water” is intended to mean that the mascara composition includes less than about 0.5% by weight of water, preferably less than about 0.1% by weight of water, relative to the total weight of the mascara composition.

According to one aspect of the present invention, the mascara composition is not in the form of an emulsion. The mascara composition may include either water phase or oil phase alone, or may include non-emulsified water phase and oil phase.

The mascara composition of the present invention is intended to be applied onto keratinous materials such as keratin fibers, in particular, eyelashes or eyebrows.

According to one aspect of the present invention, the mascara composition may have a texture suitable to be applied onto keratinous materials. The texture may be evaluated, for example, by measuring viscosity or thickness of the mascara composition. The texture may also be evaluated by experienced researchers or users by checking dragging properties or tackiness of the mascara composition.

Preferably, after the mascara composition is applied onto keratinous materials, the mascara composition is allowed to dry before subjecting to contact with other objects such as clothing and skin. According to one aspect of the present invention, the mascara composition dries within a sufficiently short time for making up keratin fibers such as eyelashes and eyebrows. Depending on the amount of the mascara composition applied onto the keratinous materials, the mascara composition may completely dry within about 15 minutes, preferably about 10 minutes, more preferably about 5 minutes, after the application. As used herein, the mascara composition is considered “completely dried” when the mascara composition is not transferred to other objects upon contacting therewith.

According to one aspect of the present invention, the mascara composition may have long-lasting properties and thus may stay on keratinous materials such as keratin fibers for a sufficient amount of time. For example, the mascara composition may stay on the keratin fibers without chipping, flaking, breaking, ripping, smudging, and/or smearing for about 24 hours or more, preferably about 48 hours or more, more preferably about 50 hours or more, without reapplication of the mascara composition.

According to another aspect of the present invention, the mascara composition may be waterproof. That is, the mascara composition may repel water when the mascara composition is exposed to water.

According to another preferred aspect of the present invention, the mascara composition is water-removable. The mascara composition, dried or undried, may be removed by exposing the mascara composition to water for a sufficient amount of time to remove the mascara composition from the surface on which the mascara composition is applied. The water may be running water, water in a container, or water absorbed in papers, towels, or cotton pads. Preferably, after the mascara composition is exposed to water, a friction force may be applied by, for example, rubbing the mascara composition with hands, fingers, papers, towels, blotters, or cotton pads. For example, keratin fibers on which the mascara composition is applied and dried may be pinched with cotton pads containing sufficient amount of water for about 20 seconds, about 15 seconds, or about 10 seconds, and then gently wiped with the cotton pads to remove the mascara composition.

Although not wishing to be bound by any theories, it is currently believed that, by appropriately contacting the mascara composition of the present invention with water for a sufficient amount of time, an emulsion may be formed. That is, after contacting the mascara composition with water, a friction force may be applied to the mascara composition to form an emulsion, by, for example, rubbing the mascara composition with hands, fingers, papers, towels, blotters, or cotton pads. The emulsion may be a water-in-oil emulsion, or an oil-in-water emulsion, depending on the composition of the mascara composition. When the mascara composition includes PEG-20 methyl glucose sesquistearate, an oil-in-water emulsion may be formed upon contacting the mascara composition with water and optionally applying a friction force. Although not wishing to be bound by any theories, it is currently believed that the formation of the emulsion may help remove the product with no need for an additional composition, such as a makeup remover.

The mascara composition of the present invention may also be removed by using a composition such as a makeup remover generally used in the cosmetic industry and/or sold as a consumer product, such as BI-FACIL®. Such composition may be used in place of water in the same way as described above. That is, keratin fibers on which the mascara composition is applied and dried may be pinched with cotton pads containing sufficient amount of a makeup-remover for about 20 seconds, about 15 seconds, or about 10 seconds, and then gently wiped with the cotton pads to remove the mascara composition.

When using a makeup remover, the mascara composition of the present invention may be removed much quicker than conventional mascara compositions, such as a composition which does not include the oxyalkylenated ether of a sugar fatty acid ester, or a mascara composition in which the oxyalkylenated ether of a sugar fatty acid ester is present in low amounts.

According to one preferred aspect of the present invention, the mascara composition has improved feel upon application. For example, the mascara composition may have comfortable and light feel upon application.

Further, according to another preferred aspect of the present invention, the mascara composition may provide improved appearance of keratinous materials upon application. For example, the keratin fibers on which the mascara composition is applied may have a defined appearance, desired intensity of colors, sufficient thickness, length and curl, and/or no noticeable balls or globs.

As described above, according to one aspect of the present invention, the mascara composition has improved cosmetic properties such as, for example, improved waterproof characteristics, improved feel upon application (for example, texture, reduced drag or tackiness), increased anti-smudging properties, increased volume properties, increased curling properties, increased curl retention properties, increased removability (from a container or a tube, or a surface of keratinous material), and/or increased long wear properties.

### (Methods)

One aspect of the present invention provides a method of making a mascara composition including mixing at least one oxyalkylenated ether of a sugar fatty acid ester and a solvent to form a mascara composition. The mixing may be carried out by stirring, shaking, grounding, or beating, with or without heating or cooling the ingredients, optionally with a stirrer, a magnetic stirrer, a shaker, a homogenizer, or any other method suitably used to mix cosmetic composition. Preferably, the mascara composition thus prepared is not in the form of an emulsion. In one aspect, the method may include mixing at least one oxyalkylenated ether of a sugar fatty acid ester with a solvent to form a waterproof mascara composition.

One aspect of the present invention provides a method of making up keratinous materials. The mascara composition described above is applied onto the keratinous materials. The keratinous materials may be skin, nail, or keratin fibers, preferably keratin fibers, in particular eyelashes and eyebrows. The mascara composition is applied onto the keratinous materials in an amount sufficient to make up the keratinous materials. To make up the keratin fibers, the mascara composition may be applied onto the keratin fibers, for example, in an amount sufficient to increase a volume and/or length of the keratin fibers.

The way by which the mascara composition is applied onto the keratinous materials is not limited. Preferably, the mascara composition is applied onto keratin fibers by a brush or a comb.

The compositions may be applied to eyelashes as needed, preferably once or twice daily, more preferably once daily and then preferably allowed to dry before subjecting to contact such as with clothing or other objects.

According to one aspect of the present invention, the mascara composition is dried by leaving the mascara composition applied onto keratinous materials for a sufficient amount of time to dry the mascara composition. The mascara composition may dry within a sufficiently short time for making up keratinous materials, in particular keratin fibers such as eyelashes and eyebrows. Depending on the amount of the mascara composition applied onto the keratinous materials, the mascara composition may completely dry within about 15 minutes, preferably about 10 minutes, more preferably about 5 minutes, after the application.

One aspect of the present invention provides a method of enhancing an appearance of eyelashes. The mascara composition described above is applied onto the eyelashes. The mascara composition is applied onto the keratinous materials in an amount sufficient to enhance the physical appearance of eyelashes. In one aspect, the mascara composition is applied onto the eyelashes in an amount sufficient to increase a volume and/or length of the eyelashes. After the mascara composition is applied, the eyelashes may look more defined, longer, thicker and/or more curled.

In the above-described methods, the mascara composition of the present invention may be applied topically to eyelashes in an amount sufficient to make up the eyelashes, or to enhance the appearance of the eyelashes.

One aspect of the present invention provides a method of improving removal properties of a cosmetic composition.

To improve removal properties of a cosmetic composition, the above-described alkoxylated sugar fatty acid ester is added during preparation of a cosmetic composition. The cosmetic composition is preferably a waterproof mascara composition. The cosmetic composition may be an emulsion, non-emulsified mixture of a water phase and an oil phase, or a water phase or an oil phase alone. Preferably, the waterproof cosmetic composition is substantially free of water, more preferably completely free of water.

The waterproof cosmetic compositions of the present invention are preferably waterproof mascara compositions.

The mixing may be carried out after the waterproof cosmetic composition is prepared. That is, the waterproof cosmetic composition may be prepared first, and then the alkoxylated sugar fatty acid ester may be mixed with the waterproof cosmetic composition. Optionally, the alkoxylated sugar fatty acid ester is mixed with all or part of ingredients of the waterproof cosmetic composition. In one aspect, the alkoxylated sugar fatty acid ester is mixed with the ingredients of the waterproof cosmetic composition during the course of preparing the waterproof cosmetic composition. In another aspect, the alkoxylated sugar fatty acid ester is mixed with all raw ingredients of the waterproof cosmetic composition.

The waterproof cosmetic composition, or the ingredients of the waterproof cosmetic composition, may be mixed with the alkoxylated sugar fatty acid ester by stirring, shaking, grounding, or beating, optionally with a stirrer, a magnetic stirrer, a shaker, a homogenizer, or any other methods suitably used to mix cosmetic compositions. The mixing may be conducted with or without heating or cooling the ingredients.

The following examples are intended to illustrate the invention without limiting the scope as a result. The percentages are given on a weight basis, unless indicated otherwise.

Notwithstanding that the numerical ranges and parameters setting forth the broad scope of the invention are approximations, the numerical values set forth in the specific examples are reported as precisely as possible. Any numerical value, however, inherently contain errors necessarily resulting from the standard deviation found in their respective measurements.

### EXAMPLES

### Mascara Compositions

Samples A, B, and C were prepared from the ingredients shown in Table 3 below.

TABLE 3

| | | A | B | C |
|---|---|---|---|---|
| Phase | INCI Name | (wt %) | (wt %) | (wt %) |
| A1 | Isododecane | 43.77 | 38.77 | 40.77 |
| | Nylon-611/Dimethicone Copolymer | 11.33 | 11.33 | 11.33 |
| | Trimethylsiloxysilicate | 12.00 | 12.00 | 12.00 |
| | PEG-20 Methylglucose | 0.00 | 5.00 | 3.00 |
| | Sesquistearate | | | |
| A2 | Talc | 3.00 | 3.00 | 3.00 |
| | Black Iron Oxide | 20.00 | 20.00 | 20.00 |
| B | Disteardimonium Hectorite | 5.80 | 5.80 | 5.80 |
| C | Propylene Carbonate | 1.90 | 1.90 | 1.90 |
| D | Pentylene Glycol | 1.00 | 1.00 | 1.00 |
| | Caprylyl Glycol | 0.50 | 0.50 | 0.50 |
| E | Panthenol | 0.50 | 0.50 | 0.50 |
| | Ceramide | 0.10 | 0.10 | 0.10 |
| | Rosa Canina Fruit Oil | 0.10 | 0.10 | 0.10 |

| | | | |
|---|---|---|---|
| TOTAL | 100.00 | 100.00 | 100.00 |

### (i) Procedure

Each of Samples A to C was prepared by the following procedure:
   1. In a main beaker, Phase A1 ingredients were added. The main beaker was placed in a water bath, and the water bath was heated to 60 to 65° C. Contents of the main beaker were mixed until all solids were dissolved.
   2. Phase A2 ingredients (pigments) were added to the main beaker and were ground for 45 minutes by using a homogenizer.
   3. After grinding the pigments, the heat for the water bath was turned off. Phase B ingredients were added to the main beaker and mixed well.
   4. Phase C ingredients were added to the main beaker and mixed well.
   5. All Phase D ingredients were added to the main beaker and mixed well.
   6. The mixing was changed to sweeper mixing. Phase E ingredients were added to the main beaker and well mixed.
   7. The batch temperature was dropped to room temperature.
      (ii) Panel testing

The removability of Samples A and B were compared by a panel testing with ten panelists. The panelists were randomly selected and consisted of ten women, ages 18 to 65, with self-perceived medium to long lashes, who use a black waterproof mascara 5 to 7 days a week, and who desired and were willing to wear a mascara composition for three days without removing or washing the eye area.

The panelists applied Samples A and B on their eyelashes at a test center in the manner they usually use to apply mascara compositions on their eyelashes. The panelists were given each of Samples A and B contained in the same type of general mascara packages labeled with a random 3-digit code to minimize bias. The panelists applied Sample A on one side of their eyelashes, and Sample B on the other side of their eyelashes. After the application, the panelist spent 72 hours at their home wearing Samples A and B without reapplying and removing the Samples, and without washing the eye areas. To evaluate the removability of Samples A and B, after 72 hours from the application, the panelists removed Samples A and B from their eyelashes using their own makeup removers.

Evaluations of Samples A and B were conducted by providing the panelists with a questionnaire including close-end questions regarding application, appearance, feel, wear, overall satisfaction, and preference.

The results are summarized in Table 4 below.

TABLE 4

| Attribute | Sample B | Sample A |
|---|---|---|
| Application | Easy to apply | |
| | Smooth application | |
| | Too fast/too slow dry | Too fast dry time (half) |
| | time (half) | |
| | Desired look achieved | Desired look achieved |
| | too slowly (few) | too slow/fast (more than |
| | | half) |
| | Transfer onto clothes/ | Transfer onto clothes/ |
| | skin (more than half) | skin (majority) |
| Brush | Easy to contour | |
| | Precise application | |
| | Just right feel on lashes | |
| | Not enough product was | Not enough product was |
| | delivered onto brush | delivered onto brush |
| | (few) | (half) |
| | Not enough/too much | Not enough/too much |
| | product delivered onto | product delivered onto |
| | lashes (more than half) | lashes (more than half) |
| | Slow lash build-up | Slow lash build-up (few) |
| | (half) | |
| | Too thick/too thin | Too thick/too thin (more |
| | (half) | than half) |
| Scent | No noticeable scent | |
| Feel | Light/heavy feel | Heavy feel (few) |
| | (acceptable) | |
| | Sticky (few) | Sticky (more than half) |
| | Comfortable | Uncomfortable (few) |
| Appearance | Too thin (few) | Too thin/too thick (half) |
| | Too short (half) | Too short/too long (few) |
| | Desired color intensity | |
| | Defined lashes | |
| | Not voluminous enough (few) | |
| | Clumping of lashes | Clumping of lashes (half) |
| | (few) | |
| | No noticeable balls/ | Noticeable balls/globs |
| | globs | (half) |
| Wear ¹ | Flaking (few: Avg. | No flaking |
| | Day 2) | |
| | Smudging (half: Avg. | Smudging (few: Avg. |
| | 1.6 Days) | Day 2) |
| | Smearing (few: Avg. Day 2) | |
| Comfort | Comfortable | |
| Removal | Easy to remove | Difficult to remove (few) |
| Overall | Average | Above Average |
| Satisfaction | | |
| while wearing | | |
| Lasting ² | 2.2 Days (Average) | 2.8 Days (Average) |
| Irritation | None | Two panelists |

| | |
|---|---|
| Overall | Below Average |
| Satisfaction | |

¹ The panelists were asked to indicate how many days after the application they noticed the flaking, smudging, and/or smearing.
² The panelists were asked to indicate how many days the mascara compositions lasted.

In Table 4, “few” indicates 3 to 4 panelists; “half” indicates 5 panelists; “more than half” indicates 6 to 7 panelists; and “majority” indicates 8 to 10 panelists.

As shown in Table 4, the panelists indicated that Sample B (the formula containing 5% PEG-20 Methylglucose Sesquistearate) had acceptable light/heavy feel and was comfortable to wear, while Sample A (the formula free from PEG-20 Methylglucose Sesquistearate) had heavy feel and was uncomfortable to wear.

In addition, the panelists indicated that Sample B formed no noticeable balls and/or globs upon application. However, half of the panelists indicated that Sample A formed noticeable balls and/or globs.

As shown in Table 4, the panelists indicated that Sample B was easy to remove, whereas Sample A was difficult to remove. None of the panelists indicated that Sample B was difficult to remove.

(iii) Evaluation of waterproof properties (Laboratory testing)

Each of Samples A, B, and C was applied on fake eyelashes three times. Each time, the Samples were applied with ten strokes, and then dried for two minutes. The eyelash samples were then dried for 1 hour.

The eyelash samples were then placed under running water at room temperature for one minute, then rubbed twice on a blotter with a pendulum motion. For the purpose of this evaluation, any visual traces of mascara residue on the blotter were considered to indicate that the composition has non-waterproof properties.

Then the eyelash samples were immersed in water for 1 hour, 6 hours and 24 hours. After each time point, the eyelash samples were rubbed 10 times on a blotter with a pendulum motion, and visually evaluated the traces in the same manner as described above.

No trace was observed on the blotter after 1 hour, 6 hours and 24 hours for all of the Samples, indicating that the mascara compositions of Samples A, B, and C were waterproof.

To further evaluate the water- and sebum-proof properties of Samples A, B, and C, Samples A, B, and C were applied on human skin and gently rubbed. All of Samples A, B, and C remained on the human skin and did not show color transfer.

(iv) Ease of removal with water (Laboratory testing)

The fake eyelash samples prepared in the above-described evaluation were used to evaluate the water-removable properties of Samples A, B, and C. Each of the eyelash samples were tightly held between a folded cotton pad containing 1 mL of water for 10 seconds, and then were gently withdrawn. The cotton pad was replaced with a newly prepared water-containing cotton pad and the same procedure was repeated until all compositions of the Samples were removed from the fake eyelashes, if possible.

All compositions of Sample A were left on the eyelash sample, indicating that Sample A was not removable with water.

All compositions of Sample B applied on the fake eyelashes were removed with water after using 16 pieces of cotton. Such results may indicate that Sample B was removable with water.

Some compositions of Sample C were removed after using 16 pieces of cotton. However, some of the compositions of Sample C were still left on the fake lashes. Such results may indicate that Sample C was partially removable with water.

The results may indicate that the mascara composition including PEG-20 methylglucose sesquistearate has water-removable properties, and that the mascara composition including at least 5% of PEG-20 methylglucose sesquistearate can be completely removed from eyelashes with water.

(v) Ease of removal with BI-FACIL® (Lab testing)

The removal properties of Samples A, B, and C with makeup removers were also evaluated using BI-FACIL®. BI-FACIL® is one of the makeup removers used in the art to remove waterproof mascara compositions. A procedure similar to that used to evaluate the water-removable properties as described in (iv) was used to evaluate the Samples' removable properties with a makeup remover. To evaluate the Sample's removable properties with BI-FACIL®, the cotton pads containing BI-FACIL® instead of water were used. The eyelash samples prepared in (iii) above were used for this evaluation.

Sample A was almost completely removed after using 8 cotton pads. However, some amounts of the composition, although minimal, still left on the fake lashes. Such results may indicate that Sample A is partially removable with BI-FACIL®.

All compositions of Sample B were removed after using 6 cotton pads. Such results may indicate that Sample B was very easy to completely remove with BI-FACIL®.

All compositions of Sample C were removed after using 8 cotton pads. Such results may indicate that Sample C was removable with BI-FACIL®.

As described above, all mascara compositions were waterproof, and the mascara composition including at least 5% of PEG-20 methylglucose sesquistearate showed particularly preferable removable properties with water and a makeup remover.

It is very easy to include PEG-20 methylglucose sesquistearate in the raw materials of mascara compositions. In addition, manufacturing processes of mascara compositions will not be changed by the inclusion of PEG-20 methylglucose sesquistearate.

1. A mascara composition, consisting of at least one oxyalkylenated ether of a sugar fatty acid ester, an iron oxide pigment, and optionally one or more of a volatile solvent, a non-volatile solvent, a filler, a dispersant, an antioxidant, a wax, Nylon-611/Dimethicone Copolymer, Trimethylsiloxysilicate, PEG-20 Methylglucose Sesquistearate, a preservative, a fragrance, and a cosmetic active agent,
wherein the mascara composition is not in a form of an emulsion.

2. The mascara composition of claim 1, wherein an amount of the oxyalkylenated ether of a sugar fatty acid ester is at least 3% by weight relative to a total weight of the mascara composition.

3. The mascara composition of claim 1, wherein the amount of the oxyalkylenated ether of a sugar fatty acid ester is at least 5% by weight relative to the total weight of the mascara composition.

4. The mascara composition of claim 1, wherein the mascara composition is substantially free of water.

5. The mascara composition of claim 1, wherein the mascara composition is waterproof.

6. The mascara composition of claim 1, wherein an alkoxy group of the oxyalkylenated ether of a sugar fatty acid ester comprises from 2 to 4 carbon atoms.

7. The mascara composition of claim 1, wherein the oxyalkylenated ether of a sugar fatty acid ester comprises an ester of at least one sugar selected from the group consisting of glucose, fructose, galactose, sucrose, maltose, lactose, and derivatives thereof.

8. The mascara composition of claim 1, wherein the oxyalkylenated ether of a sugar fatty acid ester comprises an ester of a fatty acid comprising 8 to 20 carbon atoms.

9. The mascara composition of claim 1, wherein the oxyalkylenated ether of a sugar fatty acid ester comprises 1 to 200 oxyalkylene units.

10. The mascara composition of claim 1, wherein the oxyalkylenated ether of a sugar fatty acid ester comprises an ethoxylated glucose fatty acid ester.

11. The mascara composition of claim 10, wherein the oxyalkylenated ether of a glucose fatty acid ester is methyl glucose sesquistearate comprising 20 moles of ethylene oxide.

12. A method of making-up keratinous materials, comprising:
applying the mascara composition of claim 1 onto keratinous materials in an amount sufficient to make up the keratinous materials,
wherein the mascara composition is not in a form of an emulsion.

13. (canceled)

14. (canceled)

15-17. (canceled)

18. The mascara composition of claim 1, wherein the oxyalkylenated ether of a sugar fatty acid ester is methyl glucose sesquistearate comprising 20 moles of ethylene oxide and is present in at least 5% by weight relative to the total weight of the mascara composition and wherein the mascara composition is substantially free of water.

19. The mascara composition of claim 1, wherein the iron oxide pigment is a black iron oxide pigment.

20. The mascara composition of claim 1, wherein the wax is present.

21. The mascara composition of claim 1, wherein the cosmetic active agent is present and is selected from the group consisting of an emollient, a moisturizer, a vitamin, a UV filter, a sunscreen, and a mixture thereof.
